(19) United States
(12) Patent Application Publication
(10) Pub. No.: US 2012/0196182 A1
(43) Pub. Date: Aug. 2, 2012

(54) POSITIVE ELECTRODE ACTIVE MATERIAL FOR NONAQUEOUS SECONDARY BATTERY

(75) Inventors: Masaru Yao, Ikeda-shi (JP); Hiroshi Senoh, Ikeda-shi (JP); Kazuaki Yasuda, Ikeda-shi (JP)

(73) Assignee: NATIONAL INSTITUTE OF ADVANCED INDUSTRIAL SCIENCE AND TECHNOLGOY, Tokyo (JP)

(21) Appl. No.: 13/501,158

(22) PCT Filed: Oct. 26, 2010
(86) PCT No.: PCT/JP2010/068884

§ 371 Date: Apr. 10, 2012

(30) Foreign Application Priority Data
Nov. 12, 2009 (JP) 2009-258472

(51) Int. Cl. H01M 4/60; C07C 49/753

(52) U.S. Cl. 429/213; 568/377

(57) ABSTRACT

The present invention provides a positive electrode active material for nonaqueous solvent secondary batteries, comprising, as an active ingredient, a 1,4-benzoquinone compound having lower alkoxy groups as substitutes, and a nonaqueous secondary battery comprising the positive electrode active material as a constituent. According to the invention, a nonaqueous secondary battery having a high energy density and excellent cycle characteristics can be obtained by using a positive electrode active material composed of an organic compound with a low environmental load.

### TECHNICAL FIELD

The present invention relates to a positive electrode active material for nonaqueous secondary batteries, such as lithium ion secondary batteries, and also relates to a nonaqueous secondary battery using the active material.

### BACKGROUND ART

Lithium ion secondary batteries are used as a power supply for various devices. In particular, batteries with a higher energy density are required for use in power supplies for hybrid cars, etc. Conventionally used positive electrode active materials for lithium ion secondary batteries are mainly compounds comprising heavy metal, such as lithium cobalt oxide. However, in terms of impact on the environment, active materials comprising materials with a low environmental load are desired.

Some organic compounds that are free from heavy metal have been proposed as positive electrode active materials for lithium ion secondary batteries. In particular, 1,4-benzoquinone compounds are known to undergo two-electron transfer oxidation-reduction reactions, and there have been attempts to apply them as materials that impart high capacity to batteries (see Patent Document 1). However, the actual discharge capacity is about half of the theoretical value, and sufficient substantial energy density cannot be obtained. This is presumably attributable to the low electrical conductivity of the organic active material itself, and the instability of radical species produced in association with charge and discharge. In addition, another problem is that 1,4-benzoquinone compounds are easily soluble in the electrolyte during charge and discharge, causing low cycle characteristics.

### CITATION LIST

### Patent Document

Patent Document 1: Japanese Unexamined Patent Publication No. 2008-112630

### SUMMARY OF THE INVENTION

### Problems to be Solved by the Invention

The present invention has been made in view of the above-mentioned current status of the prior art. An object of the present invention is to provide a novel positive electrode active material for nonaqueous secondary batteries, which has a high energy density and excellent cycle characteristics, and which is composed of an organic compound with a low environmental load.

### MEANS FOR SOLVING THE PROBLEMS

The present inventors conducted extensive research to achieve the above object. As a result, the inventors found that a benzoquinone compound having a specific substituent was a material with a low environmental load, which had a high initial discharge capacity and excellent cycle characteristics. The present invention has thus been accomplished.

More specifically, the present invention provides a positive electrode active material for nonaqueous secondary batteries, and a nonaqueous secondary battery, as described below:

Item 1. A positive electrode active material for nonaqueous secondary batteries, comprising, as an active ingredient, a 1,4-benzoquinone compound having lower alkoxy groups as substitutes.
Item 2. The positive electrode active material according to Item 1, wherein the 1,4-benzoquinone compound having lower alkoxy groups as substitutes is a compound represented by the following formula:

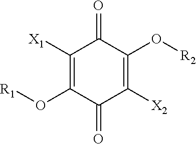

wherein R₁ and R₂ are the same or different and are each a lower alkyl group, and X₁ and X₂ are the same or different and are each a hydrogen atom or a halogen atom.
Item 3. A nonaqueous secondary battery comprising the positive electrode active material according to Item 1 or 2 as a constituent.
Item 4. The nonaqueous secondary battery according to Item 3, which comprises, as a constituent, a separator comprising a solid electrolyte.

The nonaqueous secondary battery positive electrode active material of the present invention is described in detail below.

The nonaqueous secondary battery positive electrode active material of the present invention comprises, as an active ingredient, a 1,4-benzoquinone compound having lower alkoxy groups as substitutes. The benzoquinone compound has a higher initial discharge capacity than lithium cobalt oxide widely used as a positive electrode active material for lithium ion secondary batteries. Further, the benzoquinone compound has more excellent cycle characteristics than benzoquinone compounds having no lower alkoxy group. Therefore, the use of the 1,4-benzoquinone compound as a positive electrode active material allows for the production of nonaqueous secondary batteries that have a high charge/discharge capacity and excellent cycle characteristics, as well as a low environmental load.

Although it is not necessarily clear why the 1,4-benzoquinone compound having lower alkoxy groups as substitutes has such excellent properties, the reason for this is considered to be as follows. Due to the alkoxy group of the 1,4-benzoquinone compound, radical bodies produced during charge and discharge are sterically protected and stabilized. Moreover, a one-dimensional stack structure is formed due to π-π interaction. For these reasons, it is considered that dissolution into the solvent is inhibited, and that cycle characteristics are improved. It is also considered that since the stack structure due to π-π interaction serves as a pathway of electrons during charge and discharge, electron conductivity increases, and discharge capacity becomes close to the theoretical value.

A specific example of the 1,4-benzoquinone compound having lower alkoxy groups as substitutes is a compound represented by the following formula:

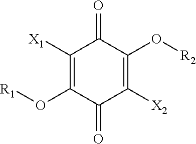

In this formula, R₁ and R₂ are the same or different and are each a lower alkyl group, and X₁ and X₂ are the same or different and are each a hydrogen atom or a halogen atom.

Among these groups, examples of the lower alkyl group include C₁₋₆ linear or branched alkyl groups, such as methyl, ethyl, n-propyl, isopropyl, n-butyl, isobutyl, tert-butyl, sec-butyl, n-pentyl, 1-ethylpropyl, isopentyl, neopentyl, n-hexyl, 1,2,2-trimethylpropyl, 3,3-dimethylbutyl, 2-ethylbutyl, isohexyl, and 3-methylpentyl. Particularly preferred among these are C₁₋₄ alkyl groups.

Examples of the halogen atom include fluorine, chlorine, bromine, and the like. Hydrogen or fluorine is particularly preferred as X₁ and X₂.

The compound represented by the above formula may be a known substance, or a substance that can be easily synthesized by dehydration reaction between dihalogenated dihydroxybenzoquinone and lower alcohol.

A nonaqueous secondary battery comprising, as a positive electrode active material, the above 1,4-benzoquinone compound having lower alkoxy groups as substitutes can be produced by a known method.

For example, the production of a lithium ion secondary battery is explained. The 1,4-benzoquinone compound is used as the positive electrode active material. The negative electrode active material is a known active material, such as metal lithium or lithium-doped carbon material (activated carbon or graphite). The electrolyte is, for example, a known electrolyte in which a lithium salt, such as lithium perchlorate (LiClO₄) or lithium hexafluorophosphate (LiPF₆), is dissolved in a solvent, such as ethylene carbonate (EC) or dimethyl carbonate (DMC). By the further use of other known battery components, a lithium ion secondary battery may be assembled according to a standard method.

In the nonaqueous secondary battery having such a structure, the use of a solid electrolyte as a separator prevents the transfer of the positive electrode active material dissolved in the electrolyte to the negative electrode, thereby greatly improving cycle characteristics. Accordingly, a nonaqueous secondary battery having a sufficient charge/discharge capacity and very excellent cycle characteristics can be obtained by using a 1,4-benzoquinone compound having lower alkoxy groups as substitutes as a positive electrode active material, and a solid electrolyte as a separator.

As a solid electrolyte, for example, for lithium ion secondary batteries, any solid electrolytes can be used without limitation as long as they have excellent lithium ion conductivity, are stable in the electrolyte used, and are able to prevent the transfer of the active material dissolved in the electrolyte. Specific examples thereof include lithium nitride, silicon, thio-LISICON, sulfide glass, and other ion-conductive ceramics; polyethylene oxide-based polymer electrolytes; and the like.

### EFFECTS OF THE INVENTION

The nonaqueous secondary battery positive electrode active material of the present invention is a material with a low environmental load, which is composed of an organic compound free from heavy metal, and which has sufficient charge/discharge capacity as well as excellent cycle characteristics. Accordingly, the use of the positive electrode active material of the present invention allows for the production of a secondary battery having lower environmental load and excellent performance.

### BRIEF DESCRIPTION OF THE DRAWINGS

FIG. 1 is a graph showing an initial discharge capacity measured in Example 1.

FIG. 2 is a graph showing cycle characteristics measured in Example 1.

FIG. 3 is a graph showing initial discharge capacities measured in Example 2.

FIG. 4 schematically illustrates a two-chamber-type sealed battery for testing produced in Example 3.

FIG. 5 is a graph showing cycle characteristics measured in Example 3.

### MODE FOR CARRYING OUT THE INVENTION

The present invention is described in more detail below with reference to Examples.

### Example 1

Using 2,5-dimethoxy-1,4-benzoquinone (Tokyo Chemical Industry Co., Ltd.) as a positive electrode active material, acetylene black as a conductive auxiliary agent, and PTFE as a binder, the active material, conductive auxiliary agent, and binder were mixed at a weight ratio of 4:5:1 to prepare a 90-μm-thick sheet. The sheet was bonded to an aluminum mesh (thickness: 110 μm) while compressing, thereby producing a positive electrode. Using this as a positive electrode material, a lithium foil as a negative electrode material, lithium perchlorate/γ-butyl lactone (1.0 mol/L) as an electrolyte, and a glass filter as a separator, a coin-type battery for testing was produced.

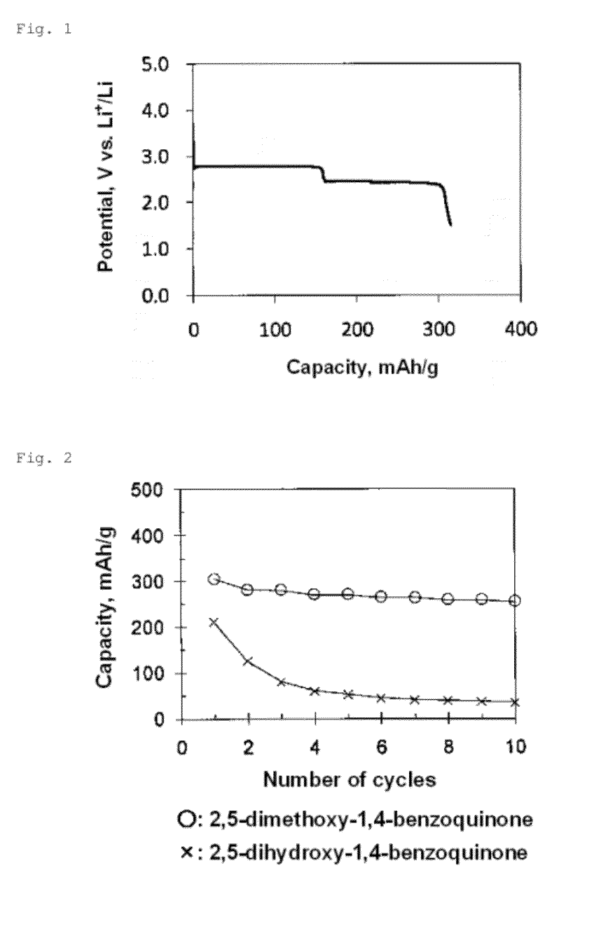

The battery was subjected to a charge/discharge test in a 30° C. atmosphere at a current density of 10 mA/g or 20 mA/g in the potential range of 1.5 to 3.4 V (vs. Li). FIG. 1 shows an initial discharge curve (current density: 10 mA/g). As is clear from FIG. 1, the discharge curve has two flat portions at potentials of 2.8 V (vs. Li) and 2.4 V (vs. Li), indicating a two-electron reaction. The initial discharge capacity was 315 mAh/g, which was twice or more than that of lithium cobaltate (140 mAh/g) generally used as a lithium ion battery positive electrode material. Thus, the battery had a high discharge capacity.

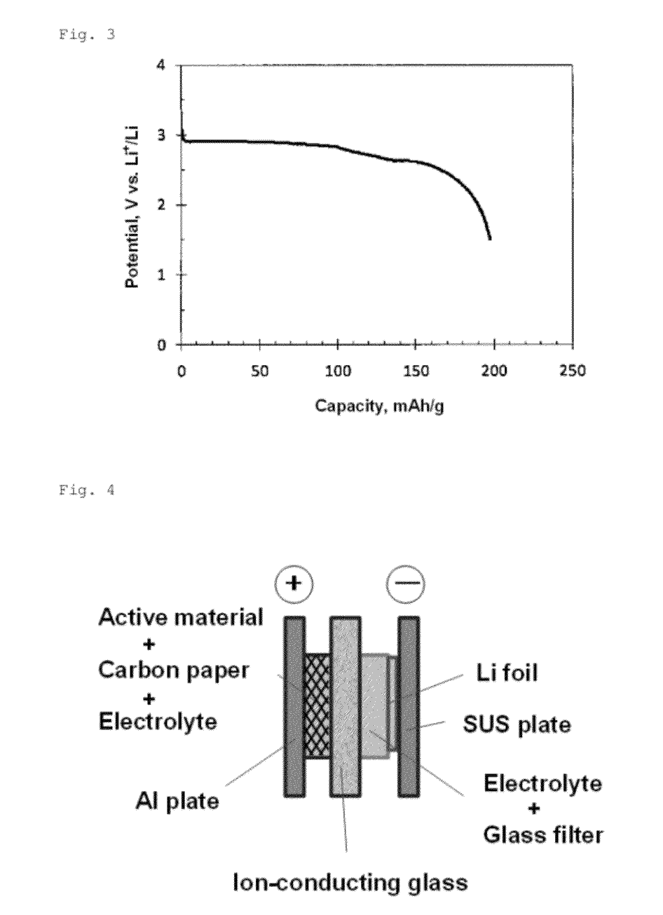

FIG. 2 is a graph showing cycle changes in the discharge capacity of the battery (current density: 20 mA/g). FIG. 2 also shows cycle characteristics of a battery using, as a positive electrode active material, 2,5-dihydroxy-1,4-benzoquinone in replace of 2,5-dimethoxy-1,4-benzoquinone.

As is clearly shown in FIG. 2, the battery comprising 2,5-dimethoxy-1,4-benzoquinone as a positive electrode active material had less capacity reduction, even when charge and discharge were repeated. Even after 10 cycles, the battery maintained a capacity of more than 250 mAh/g, and thus had excellent cycle characteristics. In contrast, as for the battery using 2,5-dihydroxy-1,4-benzoquinone as a positive electrode active material, the discharge capacity of the first cycle was about 205 mAh/g, which was about half of the theoretical capacity. The discharge capacity decreased rapidly as cycles were repeated.

### Example 2

2,5-difluoro-3,6-dimethoxy-1,4-benzoquinone was synthesized according to the method described in P. P. Sah, S. A. Peoples, Arzneimittelforschung, 1961, 11, pp. 27-33. Using this as a positive electrode active material, acetylene black as a conductive auxiliary agent, and PTFE as a binder, the active material, conductive auxiliary agent, and binder were mixed at a weight ratio of 4:5:1 to prepare a sheet. The sheet was bonded to an aluminum mesh while compressing, thereby producing a positive electrode. Using this as a positive electrode material, a lithium foil as a negative electrode material, lithium bis(pentafluoroethanesulfonyl)imide/γ-butyl lactone (3.0 mol/L) as an electrolyte, and a glass filter as a separator, a coin-type battery for testing was produced.

The battery was subjected to a charge/discharge test at a current density of 20 mA/g in the potential range of 1.5 to 3.8 V (vs. Li). FIG. 3 shows an initial discharge curve. The discharge curve had two flat portions at potentials between 2.5 to 3.0 V (vs. Li), which reflected a two-electron reaction. Moreover, the initial discharge capacity was 197 mAh/g, which was slightly lower than the theoretical capacity assuming a two-electron reaction (263 mAh/g), but greater than the discharge capacity of lithium cobaltate (140 mAh/g) generally used as a lithium ion battery positive electrode material. Furthermore, the average discharge potential of the battery was higher than that of the battery comprising 2,5-dimethoxy-1,4-benzoquinone as a positive electrode active material.

### Example 3

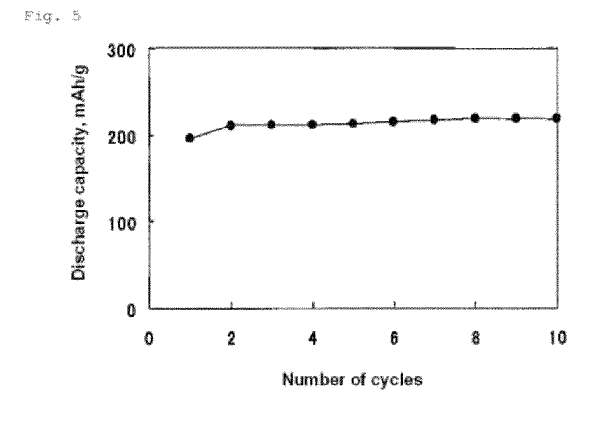

2,5-dipropoxy-1,4-benzoquinone was synthesized according to the method described in Keegstra, E. M. D.; van der Mieden, V.; Zwikker, J. W.; Jenneskens, L. W.; Schouten, A.; Kooijman, H.; Veldman, N.; Spek, A. L.; Chem. Mater., 1996, 8, pp. 1092-1105. Using this as a positive electrode active material, and an ion-conducting glass as a separator, a two-chamber-type sealed battery for testing was produced. FIG. 4 schematically illustrates the battery.

In the battery for testing shown in FIG. 4, the current collector for positive electrode was an aluminum plate, the current collector for negative electrode was a stainless steel plate, and the negative electrode material was a lithium foil. The electrolyte on the negative electrode side was lithium perchlorate/γ-butyl lactone (1.0 mol/L). The electrolyte was held in a glass filter and placed between the negative electrode (lithium foil) and the ion-conducting glass. On the other hand, the electrolyte on the positive electrode side was a solution in which 1 mg of 2,5-dipropoxy-1,4-benzoquinone (active material) was dissolved or dispersed in 50 μL of lithium perchlorate/γ-butyl lactone (1.0 mol/L). This electrolyte was impregnated into carbon paper. The carbon paper served to hold the electrolyte, in which a part of the positive electrode active material was dissolved, and the solid positive electrode active material. The carbon paper also served to improve the current-collection properties of the electrode.

The ion-conducting glass used was lithium-ion conductive glass-ceramics (LICGC; produced by Ohara Inc.), and was placed between the glass filter and the carbon paper.

The battery was subjected to a charge/discharge test at a current density of 50 μA/cm² in the potential range of 2.0 to 3.4 V (vs. Li). FIG. 5 shows the measurement results of cycle characteristics. The discharge capacity obtained was about 200 mAh/g based on active material, which was slightly less than the theoretical capacity assuming a two-electron reaction, but greater than the discharge capacity of lithium cobaltate (140 mAh/g) generally used as a lithium ion battery positive electrode material.

Moreover, this battery had very excellent cycle characteristics; almost no decrease was observed in the discharge capacity, even after 10 cycles. This is presumably because the lithium-ion conductive ceramics used as a separator was stable against the electrolyte, and had the function of preventing the passage of the active material dissolved in the electrolyte on the positive electrode side, so that the active material was prevented from moving to the negative electrode side.

1. A positive electrode active material for nonaqueous solvent secondary batteries, comprising, as an active ingredient, a 1,4-benzoquinone compound having lower alkoxy groups as substitutes.

2. The positive electrode active material according to claim 1, wherein the 1,4-benzoquinone compound having lower alkoxy groups as substitutes is a compound represented by the following formula: 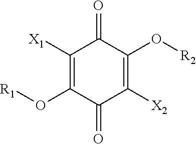
wherein R₁ and R₂ are the same or different and are each a lower alkyl group, and X₁ and X₂ are the same or different and are each a hydrogen atom or a halogen atom.

3. A nonaqueous secondary battery comprising the positive electrode active material according to claim 1 or 2 as a constituent.

4. The nonaqueous secondary battery according to claim 3, which comprises, as a constituent, a separator composed of a solid electrolyte.
